(19) United States
(12) Patent Application Publication
(10) Pub. No.: US 2015/0079583 A1
(43) Pub. Date: Mar. 19, 2015

(54) DEVICE AND METHOD FOR DETECTING A TARGET ANALYTE

(71) Applicant: Vanderbilt University, Nashville, TN (US)

(72) Inventors: Franz Baudenbacher, Franklin, TN (US); Raymond Mernaugh, Franklin, TN (US); John Mayo, Nashville, TN (US); Brad Lubbers, Madison, TN (US)

(21) Appl. No.: 14/466,305

(22) Filed: Aug. 22, 2014

Related U.S. Application Data
(60) Provisional application No. 61/868,995, filed on Aug. 22, 2013.
(60) Provisional application No. 61/877,099, filed on Sep. 12, 2013.
(60) Provisional application No. 61/879,866, filed on Sep. 19, 2013.
(60) Provisional application No. 61/883,679, filed on Sep. 27, 2013.

(51) Int. Cl. G01N 33/543; G01N 33/574; B01L 3/00; G01N 25/48

(52) U.S. Cl. CPC G01N 33/54373; G01N 25/4886; G01N 33/54306; G01N 33/57415; B01L 3/502784; B01L 2300/0819; B01L 2300/0867; B01L 2300/0896; B01L 2300/0636; B01L 2300/0645
USPC 435/5; 422/51; 436/501; 435/7.92; 435/287.2; 435/28; 435/27; 435/287.9; 435/287.1; 435/7.1; 435/7.2; 435/7.95; 435/7.93; 435/7.94; 435/6.12

(57) ABSTRACT

One aspect of the present disclosure relates to a calorimeter for detecting the presence of a target analyte in a fluid sample. The calorimeter can include a substrate, a hermetically-sealed, thermally decoupled central reaction zone associated with the substrate, at least one droplet transport region, and detection electronics. The at least one droplet transport region can be associated with the substrate and configured to merge a reagent droplet with a sample droplet including the fluid sample to form a reaction droplet in the central reaction zone. The detection electronics can be in electrical and/or thermal communication with the central reaction zone and associated with the substrate. The calorimeter can be configured to detect a heat of reaction produced by a reaction event between the target analyte and a capture reagent upon formation of the reaction droplet.

### RELATED APPLICATIONS

This application claims the benefit of U.S. Provisional Patent Application Ser. Nos. 61/868,995, filed Aug. 22, 2013, 61/877,099, filed Sep. 12, 2013, 61/879,866, filed Sep. 19, 2013, and 61/883,679, filed Sep. 27, 2013. Each of the aforementioned applications is hereby incorporated by reference in its entirety for all purposes.

### TECHNICAL FIELD

The present disclosure relates generally to diagnostic devices and methods for rapid, inexpensive, highly sensitive and specific detection of target analytes in a variety settings and industries, such as healthcare, agriculture, industry, veterinary, drug discovery, defense and homeland security.

### BACKGROUND

Currently there is no affordable point-of-care or point-of-need diagnostic platform that is rugged, durable, and provides results in minutes with a high level of sensitivity and specificity that may be operated with little training. This problem applies to diagnosing/detecting bacteria, viruses, fungi, pathogens, antibodies, biomarkers and industrial processes, as well as chemical and biological warfare agents, and numerous other uses requiring determination of the existence of a chemical reaction. Existing diagnostic platforms are very expensive in terms of both the equipment and actual test performed, require a laboratory or hospital setting, highly trained technicians, expensive infrastructure, and take from hours to days to obtain results. In many instances the required equipment is very large and requires permanent dedicated space in a facility. This current time to deliver results, lack of mobility, and the current cost of getting these results ultimately means lost lives, health, and money when utilizing current technologies.

Recent development with existing detection technologies of biological binding events and cellular activity have improved over time to the point where they are now limited by the first principles of the binding affinity of the targets or targeted pairs (e.g., antibody-antigen, etc.), or the amount of cellular activity when measuring metabolic activity. The weaker binding events and lower cellular metabolic rates are typically below the noise floor of these technologies, however, thereby rendering it hard to impossible to detect such events as those limits are reached.

### SUMMARY

One aspect of the present disclosure relates to a calorimeter for detecting the presence of a target analyte in a fluid sample. As such, the present invention may be used for determining the existence of binding or other chemical reactions that cause a thermal change, with measurement capabilities below current devices and using sample sizes that are substantially smaller than required by prior technologies. The calorimeter can include a substrate, a thermally decoupled central reaction zone associated with the substrate, at least one droplet transport region, and detection electronics. The central reaction zone may be hermetically sealed. The at least one droplet transport region can be associated with the substrate and configured to merge a reagent droplet with a sample droplet including the fluid sample to form a reaction droplet in the central reaction zone. Of course, the substrate may include pre-identified reagents in the central reaction zone and therefore eliminate the need to add or even move any droplets of the reagent. In such instances, the present invention may use disposable substrates that include specified reagents and are limited to a particular number of uses before disposal. Even if the reagent is a droplet, it may be provided with the substrate, eliminating the need to add any reagent droplets, and therefore simplifying the process for the end user to simply adding a sample to a defined area and waiting for a determination by the device. The detection electronics can be in electrical and/or thermal communication with the central reaction zone and associated with the substrate. The calorimeter can be configured to detect a heat of reaction produced by a reaction event between the target analyte and a capture reagent upon formation of the reaction droplet.

Another aspect of the present disclosure relates to a method for detecting a target analyte in a fluid sample. The method can entail the use a calorimeter that includes a substrate, a hermetically-sealed and thermally decoupled central reaction zone associated with the substrate, at least one droplet transport region associated with the substrate and configured to merge a reagent droplet with a sample droplet comprising the fluid sample, and detection electronics in electrical and/or thermal communication with the central reaction zone and associated with the substrate. One step of the method can include depositing a sample droplet within the central reaction zone. Next, the reagent droplet can be merged with the sample droplet in the central reaction zone to form a reaction droplet. The calorimeter can then detect an electronic signal generated upon formation of the reaction droplet. The electronic signal can be indicative of a heat of reaction produced by a reaction event between the target analyte and a capture reagent.

Another aspect of the present disclosure relates to a method for detecting a target analyte in a fluid sample in a point-of-care environment. The method can entail the use of a calorimeter including a substrate, a hermetically-sealed, thermally decoupled central reaction zone associated with the substrate, the central reaction zone including a temperature sensor and a surface at least partially coated with a capture reagent that specifically binds the target analyte, a first droplet transport region associated with the substrate, a second droplet transport region associated with the substrate, a third droplet transport region associated with the substrate, and detection electronics in electrical and/or thermal communication with the central reaction zone and associated with the substrate. One step of the method can include depositing a nanoliter-sized sample droplet within the central reaction zone. Next, a first droplet comprising a labeling agent coupled with a reactive moiety can be guided along the first droplet transport region until the first droplet merges with the sample droplet to form a first reaction droplet. A second droplet comprising a reaction substrate can then be guided along the second droplet transport region until the second droplet merges with the first reaction droplet to form a second reaction droplet. The calorimeter can then detect an electronic signal generated upon formation of the second reaction droplet. The electronic signal can be indicative of a heat of reaction produced by a reaction between the reactive moiety and the reaction substrate that occurs when the target analyte is present in the fluid sample.

Another aspect of the present disclosure can include a calorimeter for detecting the presence of a target analyte in a fluid sample. The calorimeter can comprise a substrate, a thermally decoupled central reaction zone associated with the substrate, at least one sample droplet transport region, and detection electronics. The at least one sample droplet transport region can be associated with the substrate and configured to merge a sample droplet comprising the fluid sample and a capture reagent to form a reaction droplet in the central reaction zone. The detection electronics can be in electrical and/or thermal communication with the central reaction zone and associated with the substrate. The calorimeter can be configured to detect a heat of reaction produced by a reaction event between the target analyte and the capture reagent upon formation of the reaction droplet.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is schematic illustration showing a calorimeter for detecting the presence of a target analyte in a fluid sample constructed in accordance with one aspect of the present disclosure;

FIG. 2 is a cross-sectional view taken along Line 2-2 in FIG. 1,

FIG. 3 is a series of photographs showing motion of a 10 nL droplet across a digital microfluidic array comprising the calorimeter in FIG. 1;

FIG. 4 is a top view showing an alternative configuration of the calorimeter in FIG. 1;

FIG. 5A is a top view showing another alternative configuration of the calorimeter in FIG. 1;

FIG. 5B is a cross-sectional view taken a long line 5B-5B in FIG. 5A

FIG. 6 is a process flow diagram illustrating a method for detecting a target analyte in a fluid sample according to another aspect of the present disclosure;

FIG. 7 is a process flow diagram illustrating a method for detecting a target analyte in a fluid sample in a point-of-care environment according to another aspect of the present disclosure;

FIG. 8 is a graph showing an assay response, using a calorimeter of the present disclosure, for 100 pg of horse radish peroxidase (HRP) in 1 nL upon the injection of 50, 100, and 200 pl of hydrogen peroxide;

FIG. 9 is a graph showing assay sensitivity, using a calorimeter of the present disclosure, with HRP and catalase for enzyme amplification (the 1/e time is directly related to the enzyme concentration and the activity of the enzyme); and

FIG. 10 is a graph showing the use of HRP for enzyme amplification to detect HERCEPTIN bound to a Her2 mimetic at therapeutic concentrations in buffer and serum using a calorimeter of the present disclosure.

### DETAILED DESCRIPTION

### Definitions

Unless otherwise defined, all technical terms used herein have the same meaning as commonly understood by one of ordinary skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the terms “about” or “approximately” can generally mean within 20 percent, preferably within 10 percent, and more preferably within 5 percent of a given value or range. Numerical quantities given herein are approximate, meaning that the term “about” or “approximately” can be inferred if not expressly stated.

As used herein, the term “subject” can refer to any warm-blooded organism including, but not limited to, human beings, pigs, rats, mice, dogs, goats, sheep, horses, monkeys, apes, rabbits, cattle, etc.

As used herein, the term “target analyte” can refer to a substance in a fluid sample capable of being detected and analyzed by the present disclosure. Target analytes can include, but are not limited to, molecules, peptides, proteins (including prions), nucleic acids, oligonucleotides, cells, microorganisms and fragments and products thereof (e.g., viruses, bacteria, fungi, fungi), enzyme substrates, ligands, carbohydrates, hormones, sugar, metabolic byproducts, cofactors, pollutants, chemical agents, small molecules, drugs, toxins, plants and fragments and products thereof, biomarkers indicative of a disease or disorder, and any substance for which attachment sites, binding members or receptors can be developed.

As used herein, the term “fluid sample” can refer to any quantity of a liquid or fluid that comprises one or more target analytes and that can be used with the present disclosure. For example, a fluid sample can be extracted from a biological sample derived from humans, animals, plants, fungi, yeast, bacteria, viruses, tissue cultures or viral cultures, or a combination of the above. In some instances, a fluid sample can comprise a bodily fluid, such as serum, blood, urine, sputum, seminal or lymph fluids. A fluid sample can also be an environmental sample, such as samples obtained from rivers or soil. A fluid sample can be first purified or partially purified, for example, and/or mixed with buffers and/or reagents that are used to generate appropriate conditions for successfully performing a method of the present disclosure.

As used herein, the term “electrical communication” can refer to the ability of a generated electric field to be transferred to, or have an effect on, one or more components of the present disclosure. In some instances, the generated electric field can be directly transferred to a component (e.g., via a wire or lead). In other instances, the generated electric field can be wirelessly transferred to a component.

As used herein, the term “thermal communication” can refer to an efficient thermal conductivity between two or more components of the present disclosure, which may or may not be in direct contact with one another (e.g., there may be one or more intervening components, structures, or elements between first and second components in thermal communication with one another). In some instances. “thermal communication” can be conductive, convective, radiative, or any combination thereof.

As used herein, the term “capture reagent” can refer to any agent that is capable of binding to, or reacting with, a target analyte. In some instances, a “capture reagent” can include an agent that is capable of specifically binding to a target analyte, i.e., having a higher binding affinity and/or specificity to the target analyte than to any other moiety. Any agent can be used as a capture reagent so long that it has the desired binding affinity and/or specificity to the target analyte. Examples of capture reagents can include, but are not limited to, antibodies, antibody fragments, recombinant antibodies and fragments thereof, native, synthetic, or recombinant peptides or proteins, peptoids, cell receptors and fragments thereof, enzymes, enzymes involved in the production of reactive oxygen species or breakdown, enzymes that catalyze a reaction leading to a product that may be of research, diagnostic or therapeutic use, p450 enzymes, glycoproteins, oligonucleotides, nucleic acids (e.g., RNA, DNA, RNA/DNA hybrids), peptide-nucleic acids, vitamins, sugars, oligosaccharides, carbohydrates, lipids, lipoproteins, small molecules, chemical compounds (e.g., hydrogen peroxide), cells, a cellular organelle, an inorganic molecule, an organic molecule, and mixtures or complexes thereof. It will be appreciated that capture reagents may also be coupled to certain substrates, such as microbeads.

As used herein, the term “heat of reaction” can refer to the heat evolved or absorbed during a chemical and/or physical reaction taking place under conditions of constant temperature and of either constant volume or constant pressure.

As used herein, the term “reaction event” can refer to any one or combination of molecular interactions between a target analyte and a capture reagent that generates or produces a heat of reaction.

As used herein, the term “in fluid communication” can refer to a fluid (e.g., a liquid) that can move from one part of the present calorimeter to another part of the calorimeter. The two or more parts of the calorimeter can be in fluid communication by being physically linked together or adjacent to each other, or the fluid communication can be mediated through another part of the calorimeter.

As used herein, the term “microcalorimeter” can refer to a calorimeter capable of detecting very small enthalpic changes (e.g., in the range of microcalories) using microliter-sized assay volumes.

As used herein, the term “nanocalorimeter” can refer to a calorimeter capable of detecting very small enthalpic changes (e.g., in the range of nanocalories) using nanoliter-sized assay volumes.

As used herein, the term “point-of-care environment” can refer to real-time diagnostic testing that can be done in a rapid time frame so that the resulting test is performed faster than comparable tests that do not employ the present disclosure. For example, an ELISA according to the present disclosure can be performed in less time than a conventional ELISA (i.e., less than about 30 minutes, preferably less than 15 minutes, and more preferably less than 10 minutes). Point-of-care environments can include, but are not limited to: emergency rooms; at a bedside; in a stat laboratory; operating rooms; hospital laboratories and other clinical laboratories; doctor's offices; in the field; or in any situation or locale where a rapid and accurate result is desired. In some instances, a subject from which a fluid sample is being assayed can be present, but such presence is not required.

As used herein, the term “reaction substrate” can refer to any substance upon which a reactive moiety can act (e.g., bind) to produce or generate a heat of reaction. In one example, a reaction substrate can include an enzymatic substrate, such as hydrogen peroxide, phosphate esters, p-nitrophenyl phosphate (PNPP), 2,2′-Azinobis[3-ethylbenzothiazoline-6-sulfonic acid]-diammonium salt (ABTS), o-phenylenediamine dihydrochloride (OPD), 3,3′,5,5′-tetramethylbenzidine (TMB), urea, and beta-galactosides (e.g., 4-methylumbelliferone b-D-galactopyranoside).

As used herein, the term “labeling agent” can refer to a compound or other agent used to label a molecule or molecules of interest, such as a capture reagent, a target analyte, and/or a reactive moiety, thereby providing a detectable signal for subsequent detection. In some instances, a labeling agent can include a compound or agent (e.g., an antibody) that specifically binds to a target analyte and includes a detectable moiety coupled thereto. The labeling agent need not itself carry a detectable moiety, but may instead be a component that is subsequently used to bind a detectable label. For example, the labeling agent can include a first specific binding partner, such as biotin, and a second specific binding partner, such as avidin or streptavidin that carries a detectable moiety.

As used herein, the term “reactive moiety” can include any agent, molecule, or compound capable of reacting with a reaction substrate to produce or generate a heat of reaction. In some instances, a reactive moiety can be coupled to a labeling agent. In one example, a reactive moiety can include an enzyme including, but not limited to, horseradish peroxidase, catalase, alkaline phosphatase, urease and beta-galactosidase.

As used herein, the terms “enzyme-linked immunosorbent assay” or “ELISA” can refer to an assay which is used to measure the concentration of an analyte (usually antibodies or antigens) in solution, ELISAs rely on the specific interaction between an epitope and a capture reagent, such as a matching antibody binding site. The antibodies used in an ELISA, for example, can be either monoclonal (derived from unique antibody producing cells called hybridomas and capable of specific binding to a single unique epitope) or polyclonal (a pool of antibodies purified from animal sera that are capable of binding to multiple epitopes. There are four basic ELISA formats: direct ELISA; indirect ELISA; sandwich ELISA; and competition or inhibition ELISA. In a direct ELISA, an antigen coated to a multiwell plate is detected by an antibody that has been directly conjugated to an enzyme. This can also be reversed, with an antibody coated to the plate and a labeled antigen used for detection. In an indirect ELISA, antigen coated to a polystyrene multiwell plate is detected in two stages or layers. First an unlabeled primary antibody, which is specific for the antigen, is applied. Next, an enzyme-labeled secondary antibody is bound to the first antibody. The secondary antibody is usually an anti-species antibody and is often polyclonal. Sandwich ELISAs typically require the use of matched antibody pairs, where each antibody is specific for a different, non-overlapping part (epitope) of the antigen molecule. The first antibody, termed the capture antibody, is coated to the polystyrene plate. Next, the analyte or sample solution is added to the well. A second antibody layer, the detection antibody, follows this step in order to measure the concentration of the analyte. Polyclonals can also be used for capture and/or detection in a sandwich ELISA provided that variability is present in the polyclonal to allow for both capture and detection of the analyte through different epitopes. If the detection antibody is conjugated to an enzyme, then the assay is called a direct sandwich ELISA. If the detection antibody is unlabeled, then a second detection antibody will be needed resulting in an indirect sandwich ELISA. A competition or inhibition ELISA is used to measure the concentration of an antigen (or antibody) in a sample by observing interference in an expected signal output. It can be based upon any of the above ELISA formats, direct, indirect, or sandwich, and as a result it offers maximum flexibility in set up. It is most often used when only one antibody is available to the antigen of interest or when the analyte is small, i.e., a hapten, and cannot be bound by two different antibodies.

Overview

The present disclosure relates generally to diagnostic devices and methods for rapid, inexpensive, and highly sensitive and specific detection of target analytes in a variety settings and industries, such as healthcare, agriculture, industry, veterinary, drug discovery, defense and homeland security. Essential to all chemical reactions, molecular interactions, and biological processes, is the transfer of energy according to the laws of thermodynamics. This energy flow must result in a change in energy and can be measured according to the first law of thermodynamics:

ΔU−Q−W;

where ΔU is the change in internal energy, Q is the heat added or taken out of the system, and W is the work performed by or on the system. In a closed system, U must remain constant, so all processes that produce work either consume or produce heat, and this heat can be measured.

The field of isothermal calorimetry deals with measuring this heat and characterizing reactions and processes based on it. The more accurately and quickly that temperature changes can be measured, the more details about the process can be elucidated. In the interest of maximizing calorimeter performance, there is a drive towards smaller sample volumes. This maximizes sensitivity and minimizes the time constant by reducing the thermal mass of the sample and measurement system. Calorimeter sensitivity can also be improved through the use of vacuum insulation and low thermal conductivity membrane materials like photo-definable Su-8. Using microfabrication techniques, nanowatt sensitivity thermopile-based sensors can be built, but traditionally suffer tradeoffs between ease of sample handing and sensitivity.

As described in more detail below, the present disclosure advantageously provides calorimeters capable of sub-nanowatt sensitivity based, in part, on the integration of digital microfluidics to allow for repeatable and efficient fluid sample delivery. Digital microfluidics provide an efficient fluid sample handling system while not adversely affecting calorimeter sensitivity. Advantageously, the combination of nanoscale calorimetry with digital microfluidics provides a scalable measurement system applicable to a number of disciplines, such as isothermal titration calorimetry for drug interaction screening, direct measurement of extracellular ATP levels, and new types of calorimetric bioassays.

Calorimeters

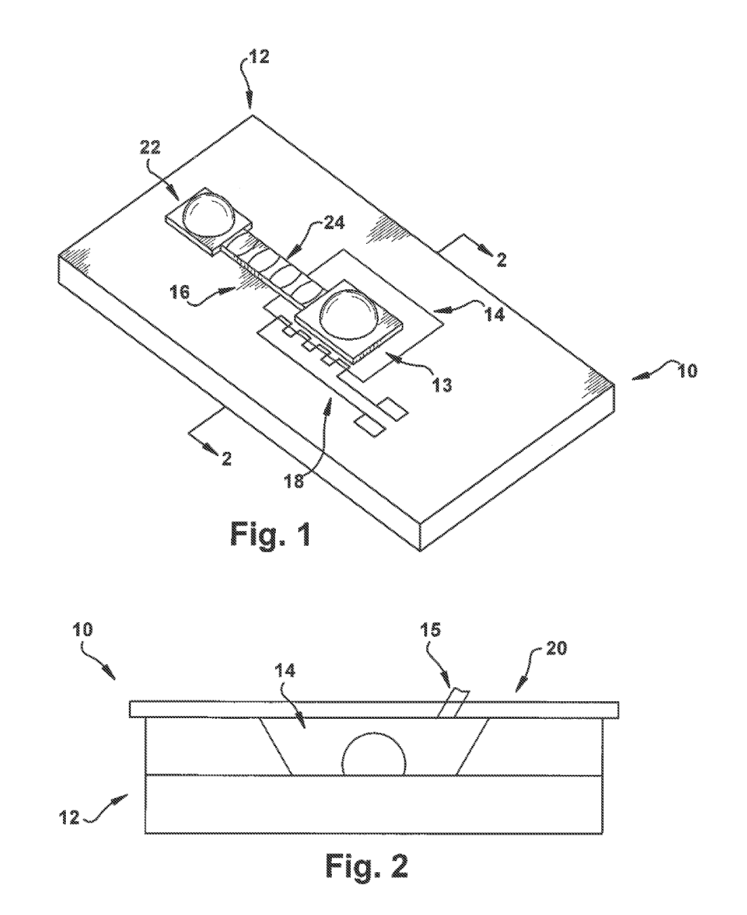

One aspect of the present disclosure can include a calorimeter 10 (FIG. 1) for detecting the presence of a target analyte in a fluid sample. The calorimeter 10 can comprise a substrate 12, a hermetically-sealed, thermally decoupled central reaction zone 14 associated with the substrate, at least one droplet transport region 16 associated with the substrate and configured to merge a reagent droplet with a sample droplet comprising the fluid sample to form a reaction droplet in the central reaction zone, and detection electronics 18 in electrical and/or thermal communication with the central reaction zone and associated with the substrate. The calorimeter 10 can be configured to detect a heat of reaction produced by a reaction event between the target analyte and a capture reagent upon formation of the reaction droplet.

Depending upon the intended application, the calorimeter 10 can be configured as a microcalorimeter or a nanocalorimeter. Advantageously, the calorimeter 10 is a simple, inexpensive, rapid, rugged, and field-deployable device that can be used under a variety of conditions and settings, such as point-of-care environments. Unlike conventional point-of-care biochemical assays, which often include fragile and expensive optics, the calorimeter 10 of the present disclosure is capable of pico- and femto-scale sensitivities (e.g., less than 100 picogram/milliliter) without the need for such optics.

In one aspect, the substrate 12 comprising the calorimeter 10 can have a single or multi-layer configuration. In some instances, one or more layers comprising the substrate 12 can include a light-sensitive material, such as an epoxy-based negative photoresist (e.g., Su-8). As described below, different components of the calorimeter 10 can be patterned on and/or integrated within the substrate 12. The substrate 12 can be prepared using microfabrication techniques, such as those described in the Examples below. One example of a multi-layered substrate 12 is illustrated in FIG. 2. Each layer of the substrate 12 can be identically or differently dimensioned when compared to other layers of the substrate. Although the substrate 12 is illustrated in FIG. 1 as having a rectangular configuration, it will be appreciated that other shapes are possible (e.g., circular, triangular, etc.).

In another aspect, the calorimeter 10 can include a hermetically-sealed, thermally decoupled central reaction zone 14 associated with the substrate 12. By “hermetically-sealed”, it is meant the area defined by the central reaction zone 14 forms an airtight seal sufficient to isolate and physically separate the central reaction zone from the ambient environment. It will be appreciated that, in some instances, the central reaction zone 14 and/or the entire calorimeter 10 need not be hermetically-sealed. It will also be appreciated that the calorimeter 10 can be substantially hermetically-sealed, meaning that only a portion of the calorimeter (e.g., only the central reaction zone 14, only the droplet transport region 16) is hermetically-sealed. By “thermally decoupled”, it is meant that a reaction droplet (discussed below) is thermally insulated from the substrate 12 and the ambient environment. Advantageously, since the calorimeter 10 measures the temperature rise associated with the reaction droplet, the thermally decoupled central reaction zone 14 provides a highly accurate environment in which to measure thermal variation since there is little or no leakage of heat, especially when compared to a system where the heat is sunk to a reservoir.

As shown in FIGS. 1-2, the central reaction zone 14 is defined by a portion of the substrate 12, at least one temperature sensor 13 (e.g., disposed on a surface of the substrate), and a sealing mechanism 20. In some instances, the temperature sensor 13 can comprise a thermopile, a resistive temperature transducer, a thermal radiation detector, or a semiconducting temperature transducer. In other instances, the temperature sensor 13 and/or a substrate surface defining the central reaction zone 14 can be at least partially coated with a capture reagent. In one example, a surface of the temperature sensor 13 can be coated with a material (e.g., gold) to promote attachment or coupling of a capture reagent thereto. In some instances, the central reaction zone 14 is free of, or does not include, a temperature sensor 13. Such a configuration of the calorimeter 10 would therefore include one or more temperature sensors 13 that are remote from the central reaction zone 14, thereby providing for a disposable substrate 12.

In another aspect, the sealing mechanism 20 can include a glass cover, a drop of oil, or a layer of material (e.g., similar or identical to the material used to form the substrate 12). The central reaction zone 14 can additionally or optionally include one or more capillary or membrane-sealed ports 15 (FIG. 2) that is/are in fluid communication with the central reaction zone and permit introduction of a fluid sample into the central reaction zone. It will be appreciated that the calorimeter 10 can include a capillary port 15 (or ports) in fluid communication with other components of the calorimeter, so long as fluid samples can be introduced into the calorimeter and ultimately disposed or located within the central reaction zone 14.

In another aspect, the substrate 12 can include at least one droplet transport region 16 associated therewith. The droplet transport region 16 can be configured to merge the reagent droplet with a sample droplet comprising the fluid sample to form a reaction droplet in the central reaction zone 14. In some instances, the droplet transport region 16 comprises a digital microfluidic array that is free of any microfluidic channels or external pumps. Digital microfluidics is based on the concept of electro-wetting of liquid droplets on a dielectric surface (EWOD). Under an applied electric field, droplets experience a reduction in surface tension in the area of the electric field. Surface tension then drives the droplets towards the area of highest surface energy. Advantageously, this effect is exploited by the present disclosure to control droplet movement electronically without the need for microfluidic channels or external pumps. It will be appreciated that, in some instances, the droplet transport region 16 can comprise an analog microfluidic array (e.g., that is free of any microfluidic channels or external pumps).

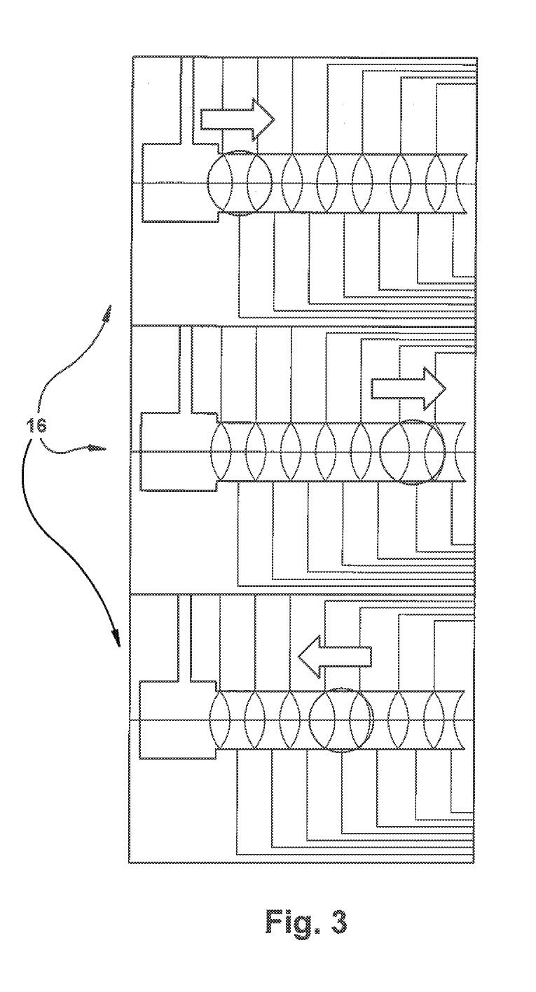

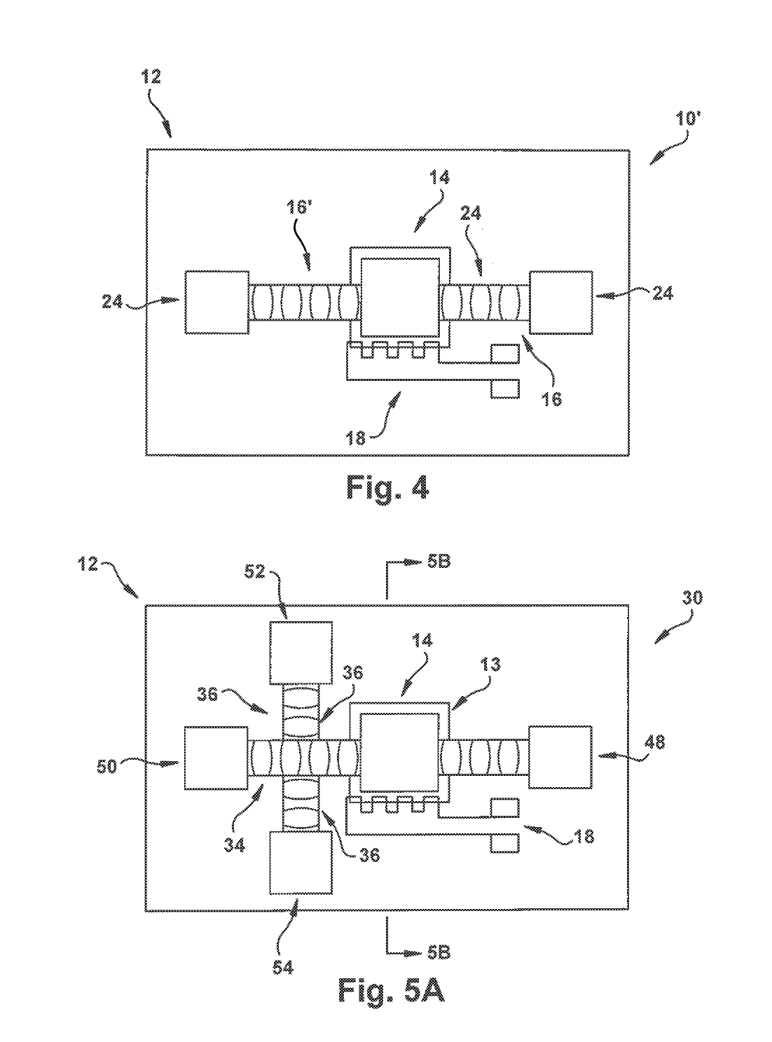

Each droplet transport region 16 can extend between the central reaction zone 14 and a loading area 22 where a reagent droplet can reside or be initially deposited. As shown in FIG. 3, each droplet transport region 16 can comprise a series of individual electrode pads arranged in a track or channel-like configuration. The calorimeter 10 can be connected to a programmable multichannel high voltage switch (not shown) to implement drop motion and/or droplet splitting. It will be appreciated that the calorimeter 10 can have any number of droplet transport regions 16, depending upon its intended use. As shown in FIG. 1, for example, the calorimeter 10 can include only a single droplet transport region 16 configured to merge a reagent droplet with a sample droplet (located in the central reaction zone 14). In this instance, the sample droplet can be deposited directly into the central reaction zone 14, whereafter all or only a portion of the reagent droplet can be guided towards the central reaction zone via the droplet transport region 16. In another example, a calorimeter 10′ (FIG. 4) can include a droplet transport region 16 configured to merge a reagent droplet with a sample droplet in the central reaction zone 14, as well as a second droplet transport region 16′ configured guide all or only a portion of the sample droplet to the central reaction zone. In other words, the sample droplet can first be deposited in a loading area 22 and then urged via the second droplet transport region 16′ to the central reaction zone 14.

One or more of the droplet transport regions 16 can include a staging area 24 where a droplet (e.g., a reagent droplet or a sample droplet) can be heated or cooled. The staging area(s) 24 can be located between the central reaction zone 14 and a loading area 22 of a droplet transport region 16. In some instances, a staging area 24 can be located between the central reaction zone 14 and a loading area 22 configured to receive the sample droplet. In such instances, all or a portion of the sample droplet can be urged along the droplet transport region 16 until it is directly over or adjacent (e.g., in direct contact with) the staging area 22. In the staging area 22, the sample droplet can be heated to a desired temperature and for a period of time sufficient to prevent or mitigate serum matrix effects, for example. In some instances, the staging area 22 can include integrated circuitry (e.g., wires embedded in a portion of the substrate 12 comprising the staging area and also in electrical communication with a power source). In other instances, the circuitry may be located under the substrate 12 (e.g., as part of a second substrate layer) such that the circuitry is not integrated with the substrate 12 and the central reaction zone 14. In such instances, all or only a portion of the substrate 12 can be heated and, when use of the substrate is finished, the substrate can be disposed of and replaced with a new substrate.

Of course, the substrate 12 may include pre-identified reagents in the central reaction zone 14 and therefore eliminate the need to add or even move any the reagent droplets. In such instances, the present disclosure may use disposable substrates 12 that include specified reagents and are limited to a particular number of uses before disposal. Even if the reagent is a droplet, it may be provided with the substrate 12, eliminating the need to add any reagent droplets, and therefore simplifying the process for the end user to simply adding a sample droplet to a defined area and waiting for a determination by the calorimeter 10.

In another aspect, the calorimeter 10 can include detection electronics 18 that are in electrical and/or thermal communication with the central reaction zone 14 and associated with the substrate 12. Detection electronics 18 can include electrical components, such as wires, capacitors, resistors, sensors, amplifiers, power sources, and the like, that may be needed for operation of the calorimeter. Detection electronics 18 can be disposed on or within the substrate(s) 12 comprising the calorimeter 10.

In another aspect, the calorimeter 10 can comprise a substrate 12, a thermally decoupled central reaction zone 14 associated with the substrate, at least one sample droplet transport region 16, and detection electronics 18. The at least one sample droplet transport region 16 can be associated with the substrate 12 and configured to merge a sample droplet comprising the fluid sample and a capture reagent to form a reaction droplet in the central reaction zone 14. The detection electronics 18 can be in electrical and/or thermal communication with the central reaction zone 14 and associated with the substrate 12. The calorimeter 10 can be configured to detect a heat of reaction produced by a reaction event between the target analyte and the capture reagent upon formation of the reaction droplet. In this configuration, the capture reagent can be present on the substrate 12 in either a liquid form (e.g., a reagent droplet) or a non-liquid form (e.g., a powder or coating). In use, the sample droplet can be guided along the sample droplet transport region 16 until the sample droplet merges with the capture reagent to form a reaction droplet (e.g., in the central reaction zone 14). Moreover, it will be appreciated that, in any configuration of the present disclosure, the capture reagent can be in either liquid or non-liquid form.

One example of a calorimeter 30 according to the present disclosure, referred to hereafter as the “Thermal ELISA”, is illustrated in FIG. 4. Conventional ELISAs use enzyme-conjugated reagents, substrates, and color developers to produce a visible or fluorescent signal that is converted—using a relatively expensive optical system—to an electronic signal indicative of analyte concentration. As described below, the Thermal ELISA 30 advantageously permits point-of-care analyte detection by detecting a heat of reaction generated either during direct binding or an enzyme-substrate reaction using a micromachined calorimeter combined with digital microfluidics to manipulate nanoliter-sized droplets. Heat is detected using thermocouples to produce an electronic signal indicative of analyte concentration in nanoliter-sized volumes. Using voltage generated by thermocouples to quantify the heat generated from direct binding (or an amplification step using an enzyme-substrate reaction), the Thermal ELISA 30 advantageously provides real-time data while also being inexpensive to make.

The Thermal ELISA 30 allows quick determination of the cessation of metabolism or cell death of target bacterial, pathogens, or cancer cells, as well as detecting the binding strength of an antibody antigen pair, other binding strengths, or the weakness of other heat producing events. As such, aspects of the present disclosure allow individuals to obtain quick feedback from doctors, and even at local drugstores as to what is going on in an individual's body. Aspects of the present disclosure could even allow an individual to visit a local drugstore to determine allergies of such an individual based upon a quick test of that individual's blood. Aspects of the present disclosure may even be configured that an individual selects a disposable substrate 12 that includes specified tests, such as reagents configured to test for allergies, particular bacteria or the like. For example, the disposable substrate 12 may have five major allergies that it can test for based on a few nanoliters of the individual's blood. As such, the person would pay the clerk for the substrate 12 and the use of the machine and put the substrate in, have a finger prick completed, which may be done automatically by the machine upon a command from the individual to start the test, and draw the blood sample automatically into the central reaction zone 14 of the disposable substrate. The present disclosure opens up a world of possibilities that previously did not exist for remote locations, drugstores, and others to provide almost real-time testing cheaply and efficiently with accurate results. The disposable nature of the substrate 12, in certain instances, may allow for a wide variety of substrates to be stocked to test for a wide variety of issues, and would eliminate the need to handle reagents by individuals. Of course, for certain common tests, the calorimeter 10 may include commonly used reagents, and self-clean the substrate and central reaction zone 14 after each use, still allowing almost autonomous use, little to no handling of the reagents by the operator or individual being tested and quick results. For a wider range of tests, certain substrates may be configured to receive a reagent at the time of test, and the sample and reagent are combined into the reaction sample.

As shown in FIG. 4, the Thermal ELISA 30 can comprise a substrate 12, a hermetically-sealed, thermally decoupled central reaction zone 14 associated with the substrate, a sample droplet transport region 32 associated with the substrate, a first droplet region transport region 34 associated with the substrate, a second droplet transport region 36 associated with the substrate, and detection electronics 18 in electrical and/or thermal communication with the central reaction zone and associated with the substrate. The central reaction zone 14 can include a temperature sensor 13 and a surface (not shown in detail) at least partially coated with a capture reagent that specifically binds a target analyte.

The substrate 12 comprising the Thermal ELISA 30 can have a single or multilayer configuration. For example, the substrate 12 comprising the Thermal ELISA 30 can be configured as shown in FIG. 2. In this multilayer configuration, a base or lower portion 38 of the Thermal ELISA 30 can include a temperature sensor 13 made of a Bi/Ti thermopile. A second layer 40 (e.g., Su-8) is placed on top of the lower portion 38. A gold pad 42 can be disposed or patterned on top of a portion of the Su-8 membrane 40. Selectively applying gold coatings to the substrate 12 permits capture of fixed amount of reagents in well defined areas of the substrate, thereby providing a less volume sensitive assay and allowing bulk loading of the Thermal ELISA 30 during manufacturing. At least part of a major surface of the gold pad 42 can be coated with a capture reagent, such as an antibody. The central reaction zone 14 can thus include a cavity 44 defined by a dielectric material 46, the Su-8 membrane 40, and a glass cover 20. A capillary port 15 (FIG. 2) (or other similar structure) for introducing a sample droplet into the central reaction zone 14 may extend through the glass cover (e.g., directly adjacent the central reaction zone).

In some instances, the substrate 12 may include a central reaction zone 14 with a base including a diamond layer. The diamond layer provides efficient transfer of heat and thermal communication to the thermocouples. In other instances, instead of or in combination with the diamond layer, a graphene layer may be used to hold the target analytes and transfer the heat efficiently and thus facilitate the thermal communication.

The calorimeter 10 (e.g., the Thermal ELISA 30) may use microliter or nanoliter-sized droplets. Of course, the calorimeter 10 may be configured to allow a large sample size to be added to the sample input area, but only move the desired size of droplet to the central reaction zone 14, whether it may be a microliter or nanoliter. The use of such small droplets has advantages in many areas that are not possible with existing technologies. Aspects of the present disclosure allow small-sized test samples, which allow a greater number of tests on the original available sample size and tests to be run that were not possible due to lack of enough sample size. For example, in criminal proceedings, generally enough sample must remain for a defendant to independently verify any findings, and too small of a sample size may eliminate the ability for the later independent verification, making the obtained evidence of little probative value. In addition, aspects of the present disclosure allow extremely small samples sizes to be collected. For example, the present disclosure is particularly useful with many blood tests, and the small sample size allows avoidance of blood draws from veins and enough sample size may be obtained from a finger prick blood test. While most people dislike finger prick tests, some companies are still marketing finger prick blood tests, because they require microliters of sample to perform the test, and can avoid the blood draw from veins. However, because current technologies to run blood test require at a minimum microliters of material, they cannot use the smallest and least invasive of technologies related to finger pricks. The smaller and less invasive finger prick tests are less painful for an individual, and are particularly useful with children. Unlike the above mentioned prior instances, which require multiple microliters of material, the present disclosure may use only nanoliters of material or less. The requirement at most for a nanoliter of material for most tests allows the use of the least invasive types of finger pricks that are barely felt by the individual, as the small sample size is still useable by the present invention. Furthermore, current technologies take, at a minimum, hours to get results, even with the larger sample size, as compared to the present disclosure which takes at most minutes to complete its analysis with a smaller sample size.

The droplet transport regions 3236 can be arranged on the substrate 12 as shown in FIG. 4. For example, the sample droplet transport region 32 can extend between a sample loading area 48 and the central reaction zone 14, the first droplet transport region 34 can extend between a first reagent droplet loading area 50 and the central reaction zone, and the second droplet transport region 36 can extend between second and third reagent droplet loading areas 52 and 54. The second and third droplet transport regions 34 and 36 can intersect one another at a common junction 56, which allows the first, second, and third reagent droplets to be selectively merged with the sample droplet to form a reaction droplet in the central reaction zone 14. Each of the droplet transport regions 3236 comprises a digital microfluidic array based on EWOD.

In one example, the Thermal ELISA 30 can have a closed design in which the temperature sensor 13 and the digital microfluidic arrays are fabricated independently on their own substrate but integrated together by placing the two halves together. In such instances, a heat flow model of the complete device can be constructed to find the optimal thermopile configuration. The EWOD control pads comprising each microfluidic digital array can occupy the bottom or lower half, while the temperature sensor 13 (e.g., thermopile) can be patterned on the top or upper half. For example, the EWOD layer can be fabricated on a freestanding Su-8 membrane (as is done for the temperature sensor 13) to reduce heat transfer to the substrate. To allow for visualization of the droplets during transport, the EWOD layer can be fabricated on a transparent glass substrate.

In some instances, the sample droplet transport region 32 can include a staging area 24 where all or only a portion of the sample droplet is heated. The staging area 24 can be located within the sample droplet transport region 32 between the central reaction zone 14 and the sample loading area 48. All or a portion of the sample droplet can be urged along the sample droplet transport region 32 until the sample droplet is directly over or adjacent (e.g., in direct contact with) the staging area 24. In the staging area 24, the sample droplet can be heated to a desired temperature and for a period of time sufficient to prevent or mitigate serum matrix effects.

In another aspect, the Thermal ELISA 30 can include detection electronics 18 that are in electrical and/or thermal communication with the central reaction zone 14 and associated with the substrate 12. Detection electronics 18 can include electrical components, such as wires, capacitors, resistors, sensors, amplifiers, power sources, and the like, that may be needed for operation of the Thermal ELISA 30. Detection electronics 18 can be disposed on or within the substrate(s) 12 comprising the Thermal ELISA 30.

Detection Methods

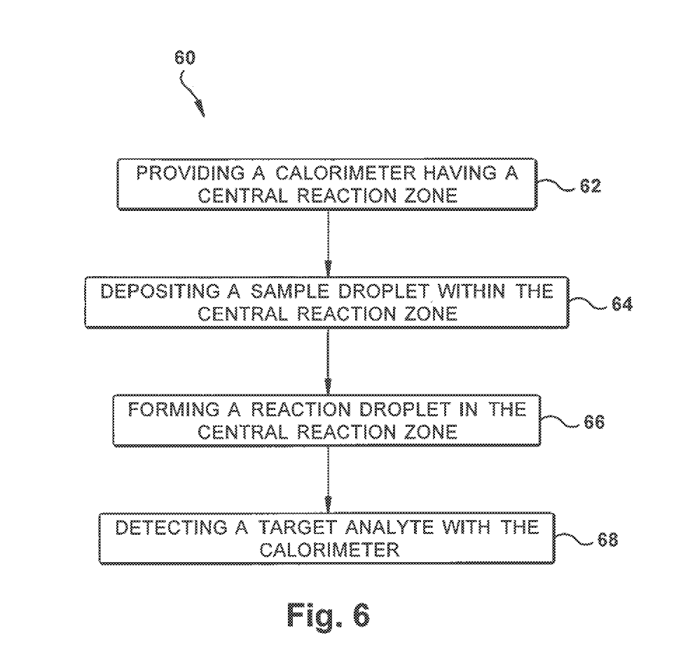

Another aspect of the present disclosure can include a method 60 (FIG. 6) for detecting a target analyte in a fluid sample. The method 60 can generally include the steps of: providing a calorimeter having a central reaction zone (Step 62); depositing a sample droplet within the central reaction zone (Step 64); forming a reaction droplet in the central reaction zone (Step 66); and detecting a target analyte with the calorimeter (Step 68). The method 60 can find use in a variety of diagnostic applications and point-of-care environments including, but not limited to, healthcare, agriculture, industry, military, and homeland security. Current point-of-care diagnostic devices and methods cannot detect target analytes (e.g., pathogens, biomarkers, chemical and biological warfare agents) within very short periods of time (e.g., within minutes) at high levels of sensitivity and specificity. Furthermore, existing diagnostic devices and methods are very expensive for the equipment and per test, require a laboratory or hospital setting, highly trained technicians, expensive infrastructure, and from hours to days to get the results.

As described in more detail below, the method 60 of the present disclosure overcomes the drawbacks of conventional diagnostic platforms and associated methods by providing a fast and efficient platform that provides point-of-care results in a short period of time (e.g., less than 10 minutes) with high sensitivity and specificity at least by virtue of its ability to detect heat in pico-joules. This pico-joule sensitivity yields the results in speed, sensitivity, specificity, and cost needed and lacking in present diagnostics. Microfluidic manipulation of sample and reagent droplets makes signals easier to detect. Additionally, unlike conventional diagnostic methods, the method 60 of the present disclosure can be performed in extreme conditions by personnel with little training.

Referring to FIG. 6, one step of the method 60 can include providing a calorimeter 10 (Step 62). In one example, the calorimeter 10 used for the method 60 can be identically or similarly constructed as the calorimeter shown in FIG. 1 and described above. Thus, the calorimeter 10 can comprise a substrate 12, a hermetically-sealed and thermally decoupled central reaction zone 14 associated with the substrate, at least one droplet transport region 16 associated with the substrate, and detection electronics 18 in electrical and/or thermal communication with the central reaction zone and associated with the substrate.

At Step 64, a sample droplet can be deposited within the central reaction zone 14. The sample droplet can be suspected of containing one or more target analytes, and can be obtained from a variety of sources. In one example, the sample droplet can include a biological sample that has been previously withdrawn from a subject (e.g., blood, serum, saliva, etc.). In another example, the sample droplet can include an environmental sample (e.g., polluted water) suspected of containing one or more target analytes. A desired volume of the sample droplet can be loaded into the calorimeter 10. In one example, the volume of the loaded sample droplet can be nanoliter-sized. In another example, the sample droplet can be microliter-sized. The sample droplet can be loaded into the calorimeter 10 via a capillary port 15 or membrane-bound port. In some instances, the sample droplet can be loaded through a capillary port 15 directly into the central reaction zone 14. In other instances, the sample droplet can be loaded onto the loading area 22 associated with the droplet transport region 16. In such instances, all or a portion of the sample droplet can be guided to the central reaction zone 14 via the microfluidic digital array comprising the droplet transport region 16. As described above, this can be achieved by applying an electric potential to the droplet transport region 16 in an amount and for a time sufficient to change the degree of hydrophilicity of the sample droplet and thereby cause all or part of the sample droplet to advance towards the central reaction zone 14.

Once the sample droplet has been loaded into the calorimeter 10, a reagent droplet can be merged with the sample droplet in the central reaction zone 14 to form a reaction droplet (Step 66). This can be achieved by applying an electric potential to the droplet transport region 16 in an amount and for a time sufficient to change the degree of hydrophilicity of the reagent droplet and thereby cause all or part of the reagent droplet to advance towards the central reaction zone 14. In one example, the reagent droplet can be nanoliter-sized. In another example, the sample droplet and/or the reagent droplet and/or the reaction droplet can be nanoliter-sized. In a further example, the sample droplet and/or the reagent droplet and/or the reaction droplet can be microliter-sized. In some instances, the reagent droplet can contain one or more capture reagents that specifically bind to and/or react with the suspected target analyte(s). In other instances, the reagent droplet can contain one or more detection elements (e.g., labeling agents, reactive moieties, reaction substrates). In such instances, one or more capture reagents may at least partially coat a surface of the central reaction zone 14. It will be appreciated that, in some instances, it may be desirable to heat the sample droplet prior to formation of the reaction droplet. In such instances, the sample droplet can be positioned about a staging area 24 and heated for a time and at a temperature sufficient to prevent or mitigate any serum matrix effects.

In some instances, the substrate 12 may include pre-identified reagent droplets (e.g., in the central reaction zone 14) and therefore eliminate the need to add or even move any the reagent droplets. In such instances, the present disclosure may use disposable substrates 12 that include specified reagents and are limited to a particular number of uses before disposal. Even if the reagent is a droplet, it may be provided with the substrate 12, eliminating the need to add any reagent droplets, and therefore simplifying the method 60 for the end user to simply adding a sample droplet to a defined area and waiting for a determination by the calorimeter 10.

Upon formation of the reaction droplet, a heat of reaction is produced or generated by the interaction of the target analyte (if present) and the capture reagent. Using the temperature sensor 13 of the calorimeter 10, heat is detected to produce an electronic signal (a potential difference or voltage) indicative of target analyte concentration (Step 68). Heat detection, and thus determination of the target analyte in the fluid sample, can be calculated using, for example, the methods described by Xu et al., Anal Chem. 80, 2728-2733 (2008) and Lubbers et al., Anal Chem. 83, 7955-7961 (2011). To extend the dynamic range of the method 60, the digital microfluidic arrays can be controlled to repeatedly pass small volumes (e.g., nanoliter-sized) of the sample droplet through the central reaction zone 14 to determine the concentration of the target analyte without driving the capture reagents into saturation. In instances where the heat of reaction is generated by an enzyme-catalyzed reaction, the reaction can be repeated multiple times by bringing in new reagents (e.g., reaction substrates) to increase sensitivity. Since the enzyme is not consumed in such a reaction, this leads to amplified heat production and thus signal generation.

It will be appreciated that the method 60 can find use in a variety of assays and applications including, but not limited to:

polymerase chain reaction (PCR)—PCR relies on thermal cycling of cycles of repeated heating and cooling of the reaction for DNA melting an enzymatic replication of the DNA. Primers containing sequences complementary to the target region along with DNA polymerase are key components to enable selective and repeated amplification. As PCR progresses, the DNA generated is itself used as a template for replication, setting in motion a chain reaction in which the DNA template is exponentially amplified. PCR can be extensively modified to perform a wide array of genetic manipulations. The calorimeter and method of the present disclosure permits rapid heating of samples (e.g., by way of laser-emitting diodes or resistors) to rapidly cycle the temperature of the samples and enable PCR. The nanoliter-sized volumes used as part of the method 60 allow for almost instant heating and cooling of the droplets and, thus, the temperature cycles needed for PCR;

antibiotic analysis—the method 60 can be used to evaluate a complete panel of antibiotics against a given bacterium to determine which antibiotic is most effective by, for example, monitoring cessation or alteration of cellular activity when the correct antibiotic is applied. This can also find use in determining whether a given bacterium is drug resistant. Further, since it is known that bacterial enzymes can break down antibiotics, the method 60 can be used to detect a thermal signature when a bacterium producing a particular enzyme is contacted with a particular antibiotic;

chemotherapy agents—the method 60 can find use in determining the correct chemotherapy agent for use against specific types of cancer. For example, a panel of chemotherapy agents can be screened against particular cancer cells to determine the effect on cancer cell activity. The cessation or alteration of cancer cell activity can be used to identify optimal chemotherapy agent(s) (or lack of one) without subjecting the subject to the side effects of trial and error;

drug screening—the pharmaceutical industry is constantly looking for new small molecule drugs to treat disease. Many new drug candidates are screened to determine if they are chemically-modified by P450 enzymes (so-called because they absorb light at a wavelength of 450 nanometers). These enzymes are present in the human liver and can metabolically alter drugs given to human patients. Additionally, microbes (e.g., bacteria, fungi, etc.) produce similar enzymes. The P450 enzymes can be costly to purify and screen, so the method 60 of the present disclosure can be used to screen for new drug candidates and thereby decrease assay costs; and

pathogenic bacteria screen—pathogenic bacteria (such as some strains of Staphylococcus aureus) produce the enzyme catalase. The catalase enzyme test is one of the key biochemical tests used to characterize a bacterial infection. The bacterium is mixed with hydrogen peroxide. If the bacterium produces catalase, then hydrogen peroxide will be enzymatically decomposed. The method 60 of the present disclosure can be performed by mixing a bacterium with hydrogen peroxide (which produces bubbles) as well as a detectable amount of heat, which can be detected by the calorimeter 10.

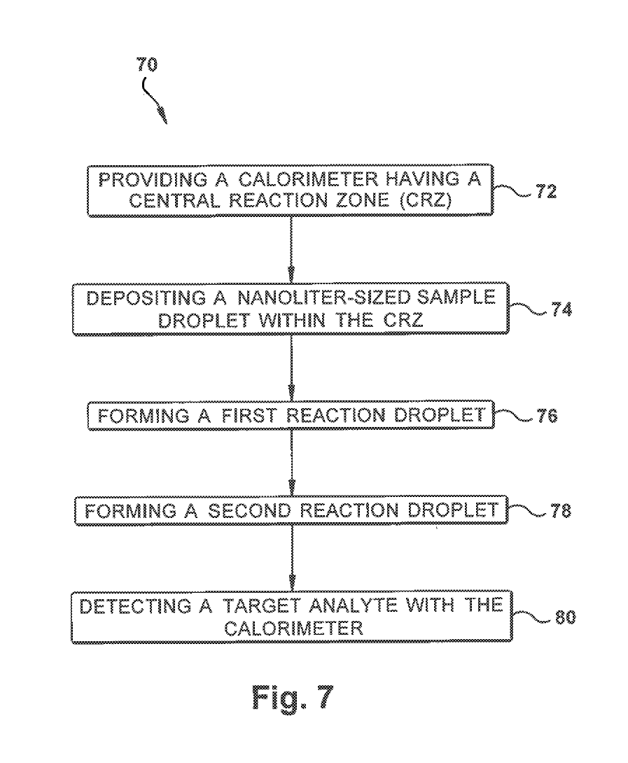

Another aspect of the present disclosure is illustrated in FIG. 7 and includes a method 70 for detecting a target analyte in a fluid sample in a point-of-care environment. The method 70 can generally include the steps of: providing a calorimeter (Step 72); depositing a nanoliter-sized sample droplet within a central reaction zone of the calorimeter (Step 74); forming a first reaction droplet (Step 76); forming a second reaction droplet (Step 78); and detecting a target analyte with the calorimeter (Step 80).

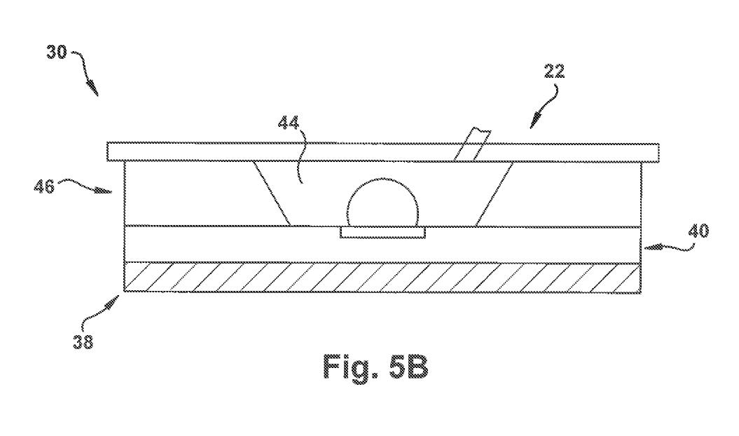

At Step 72, a calorimeter 30 as shown in FIGS. 5A-B and described above may be used for the method 70. As also as discussed above, the calorimeter 30 will be referred to throughout the description of the method 70 as the “Thermal ELISA” Although the method 70 is described below in terms of an ELISA, it will be appreciated that other assays (such as those discussed above) may also be performed.

Current ELISAs are expensive to operate, expensive to maintain, and are slow. They require highly trained technicians and are relatively fragile. They are done in a laboratory setting with expensive reagents and take from hours to days to produce results. Also, conventional ELISAs use enzyme-conjugated reagents, substrates, and color developers to produce a visible or fluorescent signal that is converted (using a relatively expensive optical system) to an electronic signal indicative of analyte concentration. The method 70 of the present disclosure, however, is advantageously based on measurement of heat generated by direct-binding events (e.g., label-free) or enzyme-conjugated reagents and substrates (e.g., as in a conventional sandwich ELISA). As described below, the method 70 provides real-time data and uses voltage generated from a calorimeter 30 to quantify the heat generated from direct binding or an amplification step using an enzyme-substrate reaction. Based on a model taking into account calorimeter characteristics, the known reaction enthalpy and enzyme activity, the heat generated can be used to directly determine the enzyme concentration (and concomitantly the analyte concentration) unaffected by the optical properties of the sample or electronic offsets. Advantageously, the nanoliter-sized volumes used with the method 70 limit sample and reagent diffusion times to seconds and thereby increase assay throughput, multiplexing and sample consumption.

At Step 74, a nanoliter-sized sample droplet can be deposited within the central reaction zone 14. The central reaction zone 14 can include a temperature sensor 13 and a surface at least partially coated with a capture reagent that specifically binds or reacts with the target analyte. The sample droplet can be suspected of containing one or more target analytes, and can be obtained from a variety of sources. In one example, the sample droplet can include a biological sample that has been previously withdrawn from a subject (e.g., blood, serum, saliva, etc.). In another example, the sample droplet can include an environmental sample (e.g., polluted water) suspected of containing one or more target analytes. The sample droplet can be loaded into the Thermal ELISA 30 via a capillary or membrane-bound port 15 that is in fluid communication with a loading area 48 of the sample droplet transport region 32. All or a portion of the sample droplet can be guided to the central reaction zone 14 via the sample droplet transport region 32. This can be achieved by applying an electric potential to the sample droplet transport region 32 in an amount and for a time sufficient to change the degree of hydrophilicity of the sample droplet and thereby cause all or part of the sample droplet to advance towards the central reaction zone 14. The target analyte, if present in the sample droplet, can specifically bind to or react with the capture reagent upon transport of the sample droplet to the central reaction zone 14.

Next, a first droplet comprising a labeling agent (e.g., an antibody that specifically binds to the target analyte) coupled with a reactive moiety (e.g., an enzyme) can be guided along the first droplet transport region 34 until the first droplet merges with the sample droplet to form a first reaction droplet (Step 76). This can be achieved by applying an electric potential to the first droplet transport region 34 in an amount and for a time sufficient to change the degree of hydrophilicity of the first droplet and thereby cause all or part of the first droplet to advance towards the central reaction zone 14. A third droplet comprising a wash solution (e.g., buffered PBS) can then be guided along the second droplet transport region 36, and part of the first droplet transport region 34, until the third droplet merges with the first reaction droplet and removes any unbound reactive moiety from the central reaction zone 14. This can be achieved by applying an electric potential to the second droplet transport region 36 in an amount and for a time sufficient to change the degree of hydrophilicity of the third droplet and thereby cause all or part of the third droplet to advance towards the central reaction zone 14.

At Step 78, a second droplet comprising a reaction substrate (e.g., specific to the enzyme comprising the reactive moiety) can be guided along a different portion of the second droplet transport region 36, and part of the first droplet transport region 34, until the second droplet merges with the first reaction droplet to form a second reaction droplet. This can be achieved by applying an electric potential to the second droplet transport region 36 in an amount and for a time sufficient to change the degree of hydrophilicity of the second droplet and thereby cause all or part of the second droplet to advance towards the central reaction zone 14. Upon merger of the second droplet with the first reaction droplet, the reactive moiety reacts with the reaction substrate and, in the process of doing so, generates or produces a heat of reaction.

Using the temperature sensor 13 of the Thermal ELISA 30, heat is detected to produce an electronic signal (a potential difference or voltage) indicative of target analyte concentration (Step 80). Heat detection, and thus determination of the target analyte in the sample droplet, can be calculated using, for example, the methods described by Xu et al., Anal Chem. 80, 2728-2733 (2008) and Lubbers et al., Anal Chem. 83, 7955-7961 (2011). The method 70 can be repeated multiple times by bringing in new reagents (e.g., reaction substrates) to increase sensitivity. Since the reactive moiety (e.g., enzyme) is not consumed in the reaction, this leads to amplified heat production and thus signal generation.

Other advantages of the method 70, besides those described above, can also include:

the use of nanoliter-sized volumes permits numerous (e.g., hundreds) of assays to be performed and thereby enhance signal-to-noise and allow multiple target analytes to be detected in a multiplexed, high-throughput rapid screening approach;

the absolute amount of bound enzyme label can be determined if the activity of the enzyme is known. In such instances, the Thermal ELISA 30 does not need any calibration and, thus, the assay can be performed to determine the activity of the enzyme just prior to the determination of the amount of bound enzyme label(s) (with the assumption of Michaelis-Menten reaction kinetics);

when reactions take place over long time periods, the detection of small amounts of bound enzymes can be enhanced if, after a sufficient time, a known excess amount of enzymes is added to the reaction volume and the amount of heat generated is measured. The amount of substrate turned over can be determined and subtracted from the amount of substrate added after the initial enzyme labeling step resulting in the amount of substrate turned over by the originally-bound enzyme labels. This way, drifts in the baseline can be eliminated and result in enhanced sensitivity;

multiple substrate injections can be averaged to enhance the signal-to-noise ratio averaging multiple injections of substrate over time;

the 1/e time constant can be directly used to determine the amount of enzyme labels bound, thereby simplifying quantification and enhancing enzyme specificity; and

signal gating can be used to eliminate crosstalk between the digital microfluidics and calorimeter signal amplification/processing.

The following examples are for the purpose of illustration only and are not intended to limit the scope of the claims, which are appended hereto.

The overall goal of the Examples was to combine nano-calorimetry with digital microfluidics to develop a quantitative rapid ELISA platform technology utilizing thermal readout strategies with picogram/milliliter sensitivity. FIGS. 5A-B represent one configuration of a nanocalorimeter (e.g., the Thermal ELISA 30) capable of point-of-care immunoassays with pictogram/milliliter sensitivities. The Thermal ELISA 30 was used to quantify HERCEPTIN antibodies (Genentech USA, Inc., San Francisco, Calif.) in human serum.

### Example 1

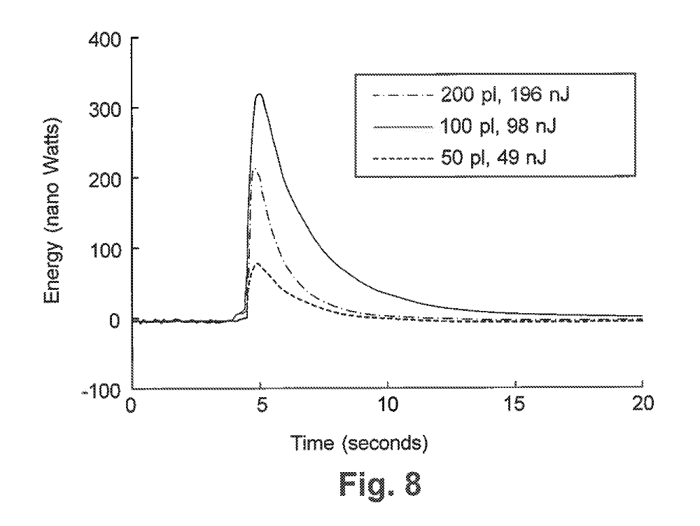

When nano-calorimetry is used to measure the heat generated by an enzyme (e.g., HRP)-catalyzed reaction, the heat generated is proportional to the reaction's substrate (e.g., H₂O₂) concentration (FIG. 8) and the time course is determined by enzyme activity, reaction volume and intrinsic device characteristics. If we select the direct-binding of IgG to protein A as a benchmark and use 45 kJ/mole heat of reaction, a heat uncertainty of 1 nJ corresponds to an IgG detection limit of 45·10⁻¹⁵ mole or the smallest mass of 720 pg based on a mass of 160 kDa for IgG. Although, there are biomarkers where a label free detection is feasible, we decided to use HERCEPTIN (Genentech USA, Inc., San Francisco, Calif.) as an exemplar model system which requires an enzyme amplification step to extend sensitivities to therapeutic relevant concentrations.

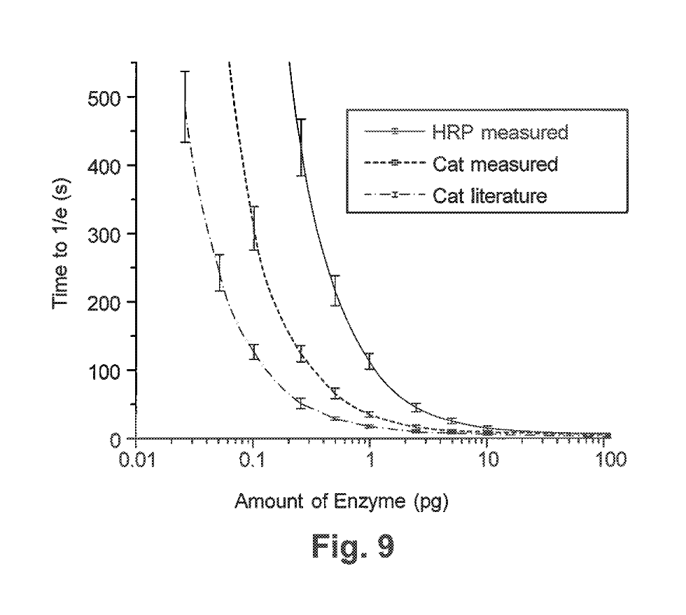

When HERCEPTIN (Genentech USA, Inc., San Francisco, Calif.) IgG is bound to the nano-calorimeter sensor surface and is detected using an HRP-conjugated anti-human IgG antibody and H₂O₂, our detection limit is <10 pg. The preliminary data (FIGS. 8-9) show that enzyme amplification increases the detection limit by at least 2-3 orders of magnitude. Currently, micropipettes are used to deliver sub nL droplets to the sample on the surface of the nano-calorimeter. This approach is not ideal, is labor intensive and difficult to automate with limited fluid handling/routing capabilities. Fluid handling and antigen/sample routing is achieved without the use of channels utilizing EWOD or digital microfluidics to move fluids across assay sensor surfaces encased in a hermetically-sealed environment, with samples being introduced into the device via capillarity using an easily penetrable port.

Digital microfluidics is based on the concept of EWOD. Under an applied electrical field, droplets experience a reduction in surface tension in the area of the electrical field. Surface tension then drives the droplets toward the area of highest surface energy. This effect can be exploited to control droplet movement electronically without the need for microfluidic channels or external pumps. FIG. 3 shows an actual sequence of imaged droplets demonstrating droplet movement along the pads of an EWOD track. Our drive electronics allowed us to move nL droplets at pad transition rates of up to 50 Hz. EWOD can also be used to reliably split and combine drops thereby allowing sequences of wash, dilution, enrichment and downstream reactions to be performed all on chip. The thermopile, Su-8 layers/membrane, and backside etch dimensions are all controlled by a photomask layout. The major fabrication steps and the flip chip assembly for the digital nano-calorimeter are described below.

Calorimeter Fabrication

Openings in the backside of a double-sided silicon nitride (SiN) coated Si <100> wafer are made first. Dry etching of SiN and patterning is carried out using Shipley's 31813. The exposed Si is anisotropically etched in 30% w/w KOH at 80° C. Su-8 2002 is spun onto the surface at 3000 rpm to generate a 2 μm thick Su-8 membrane layer (step 2). A 200 nm Ti layer is deposited using an Innotech e-beam deposition/ion-mill system and patterned with S1813 (step 3). Next a 400 nm Bi layer is applied and patterned as in step 3 (step 4). This is thicker than the Ti due to the high resistivity of Bi and a direct increase in thermopile noise with resistance. A 100 nm silicon dioxide passivation layer is then applied to prevent oxidation and damage to the thermopile (step 5). Next a 10 nm Au layer is applied to provide the ground plane for the digital microfluidics (step 6). The contact pads are masked off during deposition during step. The SiN under the membrane is removed using Reactive Ion Etch (RIE) (step 7). The wafer is then diced and each chip sealed with a lid containing the digital microfluidics (step 8).

Digital Microfluidics Fabrication

The EWOD layer is also fabricated on a freestanding Su-8 membrane on a SiN substrate using the same techniques as above. The electrodes are patterned in Ti and an additional 2 μm Su-8 layer spun on top of the electrodes to form the dielectric layer. Reagent reservoirs are etched into the EWOD substrate at the same time as the free-standing Su-8 membrane is formed. The spacing of the reservoirs can be adapted to the 384 or 1536 micro-well format to make use of commercially available robotic liquid handlers for automated reagent loading.

Digital Nano-Calorimeter Flip-Chip Assembly

Spacers to define the gap between the EWOD layer and the calorimeter are created using Su-8 in thicknesses ranging between 10 and 50 μm. Then, 1% Teflon AF (DuPont) is spun on at 1000 rpm to form a hydrophobic layer. This is baked at 260° C. to dry and reflow the fluoropolymer and seal the device. Reagent is loaded into reservoirs and covered by oil to prevent evaporation and hermetically seal the device. The device is connected to a programmable multichannel high voltage switch to implement drop motion and droplet splitting. To perform ELISA, multiple fluid reservoirs for samples, enzyme-labeled antibodies, and wash buffers are present on the chip.

### Example 2

Model antigen/antibody and antibody/peptide systems are used that involve the use of well-defined monoclonal, Fab and single chain fragment variable (scfv) recombinant antibody fragments or peptides to identify antibody (i.e., IgG, Fab or scfv) or peptide format that results in enhanced assay sensitivity and specificity when used in assays for serum samples. The MOB antibody binds to and captures rabbit IgG from solution. A10B monoclonal IgG, Fab or scfv antibodies are immobilized on the gold sensor surfaces using either passive adsorption, biotinylated and bound to avidin/streptavidin, or engineered (e.g., cysteine amino acid) sulfhydryl (—SH) group. A direct ELISA using rabbit IgG conjugated to peroxidase (rabbit IgG/HRP) and H₂O₂ is used to determine if the A10B capture antibody retains biological activity. For an indirect or sandwich-based ELISA, rabbit IgG captured by A10B is detected using commercially available goat anti-rabbit IgG conjugated to enzymes (e.g., peroxidase, alkaline phosphatase, beta-galactosidase, etc.) and a suitable substrate to determine assay sensitivity and specificity.

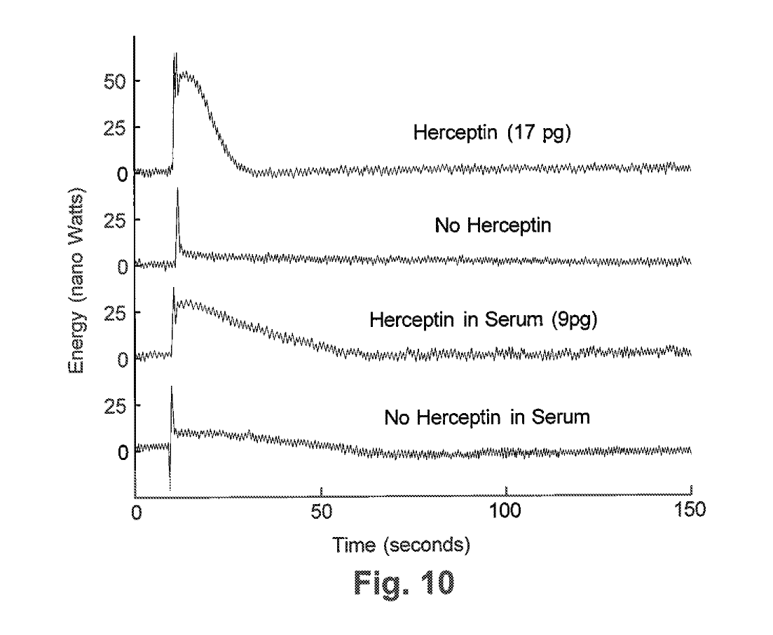

A number of A10B scfv mutants have been engineered to display amino acids (e.g., cysteine) that can be used to directly couple the A10B scfv to surfaces (e.g., gold surfaces) as a self-assembled monolayer (SAM) to capture rabbit IgG out of samples. The A10B scfv and IgG monoclonal antibodies have also been used in assays involving SPR and/or mass spectrometry. The CH-19 synthetic peptide is a HER2 mimetic to which HERCEPTIN binds. Our preliminary data were obtained utilizing biotinylated CH-19 peptide immobilized onto an avidin-coated gold sensor surface and an HRP-conjugated anti-human IgG as secondary label. Subsequently, H₂O₂ was used to detect HERCEPTIN spiked into buffer and pooled human normal serum samples using nano-calorimetry (FIG. 10). We have already procured well-annotated and de-identified normal and breast cancer patient serum/plasma samples from commercial sources. The CH-19 peptide is used to detect and quantify HERCEPTIN in HERCEPTIN-treated breast cancer patient samples.

Sample/Serum Treatment

Serum matrix effects are components of serum (e.g., anti-antibodies) that can bind to and interfere with reagents (e.g., synthetic peptides) used in immunoassays to detect analytes (e.g., HERCEPTIN). Heat is used to treat and disrupt/denature components in human serum (i.e., serum matrix effects such as those involving anti-antibodies) that can interfere with antigen/antibody interactions. The 2B4 scfv is a high affinity scfv that can be used to specifically capture heat-denatured HERCEPTIN from solution. Serum samples or a negative control (e.g., Bevacizumab/Avastin) is briefly heated from 65-90° C. for several seconds then passed over a sensor surface (of the device) bearing avidin/streptavidin to which biotinylated 2B4 scfv or a negative control scfv (e.g., A10B scfv) have been immobilized. Bound HERCEPTIN is detected using commercially available peroxidase conjugated anti-human IgG and H₂O₂.

Enzyme Amplification

The above data demonstrate that we can detect the heat generated by HRP using H₂O₂ as a substrate within seconds in nano-liter droplets. Horseradish-peroxidase is very often linked to secondary antibodies in conventional colorimetric ELISAs. However, other enzyme systems are available and tested for suitability in the calorimeter of the present disclosure. Catalase, for example, has one of the highest turnover numbers of all the enzymes converting peroxide. A comparison of the thermal response between horseradish peroxidase and catalase demonstrating picogram sensitivities is shown in FIG. 9. Other enzyme and substrates can include: alkaline phosphatase with p-nitrophenyl phosphate as the substrate; beta-galactosidase with 4-methylumbelliferone β-D-galactopyranoside as the substrate; and urease with urea as the substrate. All enzymes can be coupled to antibodies using commercially available crosslinking agents. For each of the enzyme and substrate systems, optimal enzyme:antibody conjugation ratios, substrate concentrations and the limit of detection for each enzyme when coupled to the same antigen-specific antibody and used in the calorimeter of the present disclosure to detect the same antigen are determined.

Determination of Calorimeter Assay Sensitivity

Samples (e.g., human serum samples containing an antigen or analyte such as the HERCEPTIN therapeutic McAb) are introduced into the calorimeter via a capillary port 15. Digital microfluidics is used to transport samples from the port to a staging site (e.g., a site containing an immobilized anti-HERCEPTIN capture antibody) and reagents (e.g., wash buffers, enzymeconjugated antibodies and substrates) to carry out the ELISA reactions. Heat is generated from the antibody-conjugated enzyme/substrate reaction at the staging site to produce an electronic signal—the magnitude and temporal profile of the signal is indicative of antigen (e.g., HERCEPTIN) presence and can be directly converted into a concentration (FIG. 9). The detection as well as the capture step can be repeated multiple times in order to increase the calorimeter sensitivity by bringing new substrate and sample to the central reaction zone. Digital microfluidics can also be used to transport serum/plasma samples to a pre-processing area where samples can be briefly heated (under controlled conditions to avoid evaporation) to disrupt serum matrix effects (e.g., anti-antibodies) to further enhance assay specificity and sensitivity.

From the above description of the invention, those skilled in the art will perceive improvements, changes and modifications. For example, it will be appreciated that components of the calorimeter (e.g., the temperature sensor 13 and the digital microfluidics) can be fabricated on the same surface of the substrate to eliminate flip chip assembly, cross plane electrical connections, and enhance sample visualization lead to a reduction of fabrication costs and increased yield. Such improvements, changes, and modifications are within the skill of the art and are intended to be covered by the appended claims. All patents, patent applications, and publication cited herein are incorporated by reference in their entirety.

The following is claimed:

1. A calorimeter for detecting the presence of a target analyte in a fluid sample, the calorimeter comprising:
a substrate;
a thermally decoupled central reaction zone associated with the substrate;
at least one droplet transport region that is associated with the substrate and configured to merge a reagent droplet and a sample droplet comprising the fluid sample to form a reaction droplet in the central reaction zone; and
detection electronics in electrical and/or thermal communication with the central reaction zone and associated with the substrate;
wherein the calorimeter is configured to detect a heat of reaction produced by a reaction event between the target analyte and a capture reagent upon formation of the reaction droplet.

2. The calorimeter of claim 1, wherein the central reaction zone is hermetically-sealed.

3. The calorimeter of claim 1, being at least one of a microcalorimeter and a nanocalorimeter.

4. The calorimeter of claim 1, wherein each of the reagent droplet, the sample droplet, and the reaction droplet is nanoliter-sized.

5. The calorimeter of claim 1, wherein each of the reagent droplet, the sample droplet, and the reaction droplet is microliter-sized.

6. The calorimeter of claim 1, wherein the reagent droplet includes the capture reagent.

7. The calorimeter of claim 1, wherein the central reaction zone further includes a temperature sensor.

8. The calorimeter of claim t wherein the central reaction zone does not include a temperature sensor.

9. The calorimeter of claim 7, wherein the central reaction zone has a major surface at least partially coated with the capture reagent.

10. The calorimeter of claim 1, wherein the temperature sensor further comprises at least one of a thermopile, a resistive temperature transducer, a thermal radiation detector, or a semiconducting temperature transducer.

11. The calorimeter of claim 1, wherein the at least one droplet transport region comprises a digital microfluidic array.

12. The calorimeter of claim 1, wherein the at least one droplet transport region comprises an analog microfluidic array.

13. The calorimeter of claim 11, wherein the at least one droplet transport region is free of any microfluidic channels or pumps.

14. The calorimeter of claim 1, further comprising:
a second droplet transport region that is associated with the substrate and configured to merge a second reagent droplet with the sample droplet; and
a third droplet transport region configured to receive the sample droplet and guide the sample droplet to the central reaction zone.

15. The calorimeter of claim 1, further comprising circuitry integrated with the central reaction zone and the substrate, the circuitry being configured to heat the sample droplet and/or apply an electric field to prevent or mitigate sample matrix effects.

16. The calorimeter of claim 1, further comprising circuitry, which is neither integrated with the central reaction zone nor the substrate, being configured to heat at least a portion of the substrate and thereby render the substrate disposable.

17. The calorimeter of claim 1, being adapted for use in a point-of-care environment.

18. The calorimeter of claim 1, wherein the substrate is disposable.

19. A method for detecting a target analyte in a fluid sample, using a calorimeter comprising a substrate, a thermally decoupled central reaction zone associated with the substrate, at least one droplet transport region associated with the substrate and configured to merge a reagent droplet with a sample droplet comprising the fluid sample, and detection electronics in electrical and/or thermal communication with the central reaction zone and associated with the substrate, the method comprising the steps of:
depositing a sample droplet within the central reaction zone;
merging the reagent droplet and the sample droplet in the central reaction zone to form a reaction droplet; and
detecting, with the calorimeter, an electronic signal generated upon formation of the reaction droplet, the electronic signal being indicative of a heat of reaction produced by a reaction event between the target analyte and a capture reagent.

20. The method of claim 19, wherein each of the reagent droplet, the sample droplet, and the reaction droplet is nanoliter-sized.

21. The method of claim 19, wherein each of the reagent droplet, the sample droplet, and the reaction droplet is microliter-sized.

22. The method of claim 19, being performed in a point-of-care environment.

23. The method of claim 19, wherein said merging step further includes selectively applying an electric potential to the at least one droplet transport region in an amount and for a time sufficient to change the degree of hydrophilicity of the reagent droplet and thereby split and/or advance the reagent droplet towards the central reaction zone.

24. The method of claim 19, the calorimeter being at least one of a microcalorimeter and a nanocalorimeter.

25. A method for detecting a target analyte in a fluid sample in a point-of-care environment, using a calorimeter comprising a substrate, a hermetically-sealed, thermally decoupled central reaction zone associated with the substrate, the central reaction zone including a temperature sensor and a surface at least partially coated with a capture reagent that specifically binds the target analyte, a first droplet transport region associated with the substrate, a second droplet transport region associated with the substrate, a third droplet transport region associated with the substrate, and detection electronics in electrical and/or thermal communication with the central reaction zone and associated with the substrate, the method comprising the steps of:
depositing a nanoliter-sized sample droplet within the central reaction zone;
guiding, along the first droplet transport region, a first droplet comprising a labeling agent coupled with a reactive moiety until the first droplet merges with the sample droplet to form a first reaction droplet;
guiding, along the second droplet transport region, a second droplet comprising a reaction substrate until the second droplet merges with the first reaction droplet to form a second reaction droplet; and
detecting, with the calorimeter, an electronic signal generated upon formation of the second reaction droplet, the electronic signal being indicative of a heat of reaction produced by a reaction between the reactive moiety and the reaction substrate that occurs when the target analyte is present in the fluid sample.

26. The method of claim 25, wherein the reactive moiety is an enzyme.

27. The method of claim 25, further comprising the step of:
guiding, along the third droplet transport region, a third droplet comprising a wash solution until the third droplet merges with the first reaction droplet and removes any unbound reactive moiety from the central reaction zone.

28. A calorimeter for detecting the presence of a target analyte in a fluid sample, the calorimeter comprising:
a substrate;
a thermally decoupled central reaction zone associated with the substrate;
at least one sample droplet transport region that is associated with the substrate and configured to merge a sample droplet comprising the fluid sample and a capture reagent to form a reaction droplet in the central reaction zone; and
detection electronics in electrical and/or thermal communication with the central reaction zone and associated with the substrate;
wherein the calorimeter is configured to detect a heat of reaction produced by a reaction event between the target analyte and the capture reagent upon formation of the reaction droplet.
